(19) United States
(12) Patent Application Publication
(10) Pub. No.: US 2024/0082181 A1
(43) Pub. Date: Mar. 14, 2024

(54) Method for Treating Blood Cancers

(71) Applicant: Lantern Pharma Inc., Dallas, TX (US)

(72) Inventors: Jianli Zhou, Dallas, TX (US); Kishor Bhatia, Iowa, MD (US); Aditya Kulkarni, Dallas, TX (US)

(73) Assignee: Lantern Pharma Inc., Dallas, TX (US)

(21) Appl. No.: 18/500,032

(22) Filed: Nov. 1, 2023

Related U.S. Application Data
(63) Continuation of application No. PCT/US2022/072091, filed on May 3, 2022.
(60) Provisional application No. 63/183,519, filed on May 3, 2021.

(51) Int. Cl. A61K 31/17; A61K 45/06; A61P 35/00; A61P 35/02

(52) U.S. Cl. CPC A61K 31/17; A61K 45/06; A61P 35/00; A61P 35/02

(57) ABSTRACT

A method for treating a subject in need thereof having a bone or blood cancer, or a cancer that metastasizes to bone that includes administering to the subject an effective amount of hydroxyureamethyl acylfulvene.

### CROSS-REFERENCE TO RELATED APPLICATIONS

This application claims the benefit of U.S. Provisional Patent Application No. 63/183,519 filed May 3, 2021, which is incorporated by reference herein in its entirety.

### TECHNICAL FIELD

This application relates to a cancer treatment, and more particularly to a method for treating blood cancers.

### BACKGROUND

Blood cancers affect blood cells and bone marrow—the spongy tissue inside bones where blood cells are made. These cancers change the way blood cells behave and how well they work. There are three major types of blood cancers: Leukemia, Lymphoma and Myeloma. These cancers cause bone marrow and the lymphatic system to make defective blood cells. They all affect different subtypes of blood cells, and they act in different ways. If discovered early, blood cancer could be treated by chemotherapy, targeted therapy or surgery, which would be ideal especially if the tumor and cancerous cells have not spread and can be removed cleanly.

A blood cancer prognosis varies based on the subtype and other factors, including overall health, age and response to treatment. Although great improvements have occurred in the area of blood cancer treatment, the overall 5-year survival of blood cancer is 70%. In addition, some patients especially elderly patients are unable to tolerate high-intensive chemotherapy or stem-cell transplant surgery, leaving them very few options for cancer treatment. Resistance or relapse to standard cancer therapies is also common. For example, 50% of Chronic myeloid leukemia (CML) patients treated with imatinib eventually develop resistance or intolerance. Some blood cancer such as mantle cell lymphoma is incurable, meaning that patients would eventually relapse from all the available treatment and run out of options. There are also rare blood cancers that remain very challenging to treat. For example, there's no established standard of care for double-hit lymphoma and the median survival time is only 5-months.

Accordingly, there is a need for improved treatments for blood cancers, including double-hit lymphoma.

### SUMMARY

One aspect of this application includes a method for treating patients with blood cancers in which hydroxyurea methyl acylfulvene or salt thereof is administered in a therapeutically effective amount to a subject in need thereof with blood cancers. Blood cancers can include mantle cell lymphoma (MCL), double-hit lymphoma (DHL), Burkitt's Lymphoma, Anaplastic large cell lymphoma (ALCL), Multiple Myeloma (MM), acute myeloid leukemia (AML), acute lymphoblastic leukemia (ALL), and chronic myeloid leukemia (CML).

Another aspect includes a pharmaceutical composition having an effective amount of hydroxyureamethyl acylfulvene, or a pharmaceutically acceptable salt thereof; and (b) at least one pharmaceutically acceptable carrier. One hydroxyureamethyl acylfulvene, labeled Compound 1 is shown as follows:

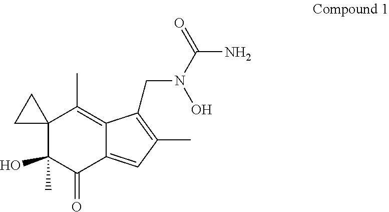 [SMILES: CC1=C(CN(O)C(N)=O)C2=C(C)C3(CC3)[C@](C)(O)C(=O)C2=C1]

Another aspect includes the administration of a second anticancer agent that may include DNA damage agents, glucocorticoids, immunomodulatory drugs (IMiDs), BCL2 inhibitors, Bruton's tyrosine kinase inhibitors, PARP inhibitors, and/or proteasome inhibitors.

Another aspect includes the administration of hydroxyureamethyl acylfulvene to treat blood cancers orally, topically, intranasally, systemically, intravenously, subcutaneously, intraperitoneally, intradermally, intraocularly, iontophoretically, transmucosally, or intramuscularly.

Another aspect includes a method in which the blood cancer is lymphoma or myeloma.

Another aspect includes a method in which the blood cancer is mantle cell lymphoma (MCL), double-hit lymphoma (DHL), Burkitt's Lymphoma, Anaplastic large cell lymphoma (ALCL), or Multiple Myeloma (MM).

Another aspect includes a method in which hydroxyureamethyl acylfulvene is administered in conjunction with administering to the subject in the need thereof a second anti-cancer agent.

Another aspect includes a method in which the second anti-cancer agent is selected from DNA damage agents, glucocorticoids, immunomodulatory drugs (IMiDs), BCL2 inhibitors, Bruton's tyrosine kinase inhibitors, spironolactones, PARP inhibitors, and/or proteasome inhibitors.

Another aspect includes a method in which the subject in need thereof is simultaneously being treated with another therapy to treat acute myeloid leukemia, acute lymphoblastic leukemia, myelodysplastic syndrome, a myeloproliferative disorder, or chronic myelogenous leukemia.

Another aspect includes a method in which the subject in need thereof is human.

Another aspect includes a method in which the blood cancer is mantle cell lymphoma (MCL), double-hit lymphoma (DHL), Burkitt's Lymphoma, Anaplastic large cell lymphoma (ALCL), Multiple Myeloma (MM), acute myeloid leukemia (AML), acute lymphoblastic leukemia (ALL), and/or chronic myeloid leukemia (CML).

Another aspect includes a method for treating a subject in need thereof having a blood cancer, or a cancer that metastasizes to bone. The method comprising administering to the subject an effective amount of hydroxyureamethyl acylfulvene or Compound 1.

### DESCRIPTION OF THE FIGURES

FIG. 1 shows a table demonstrating the effectiveness and selectivity of the cytotoxic activity of Compound 1 using a panel cell lines.

### DETAILED DESCRIPTION

This application includes a method for treating patients with blood cancers in which hydroxyureamethyl acylfulvene or salt thereof is administered in a therapeutically effective amount to a subject with a blood cancer. In certain embodiments, the blood cancer may be mantle cell lymphoma (MCL), double-hit lymphoma (DHL), Burkitt's Lymphoma, Anaplastic large cell lymphoma (ALCL), Multiple Myeloma (MM), acute myeloid leukemia (AML), acute lymphoblastic leukemia (ALL), and/or chronic myeloid leukemia (CML). The term “blood cancer” can refer to a disease including abnormal cell growth with the potential to spread to other parts of the body. Such blood cancer includes mantle cell lymphoma (MCL), double-hit lymphoma (DHL), Burkitt's Lymphoma, Anaplastic large cell lymphoma (ALCL), Multiple Myeloma (MM), acute myeloid leukemia (AML), acute lymphoblastic leukemia (ALL), and chronic myeloid leukemia (CML).

In a specific embodiment, the structure of hydroxyureamethyl acylfulvene (termed LP-284 by Lantern Pharma Inc., which shifts light positively (has a positive optical rotational angle), is shown below:

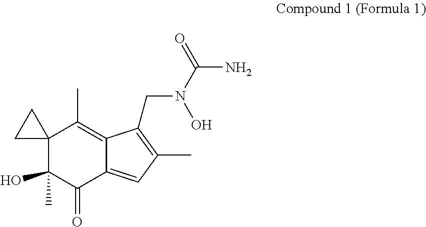 [SMILES: CC1=C(CN(O)C(N)=O)C2=C(C)C3(CC3)[C@](C)(O)C(=O)C2=C1]

In another specific embodiment, the structure of hydroxyureamethyl acylfulvene (termed LP-184 by Lantern Pharma Inc., which shifts light positively (has a negatively optical rotational angle), is shown below:

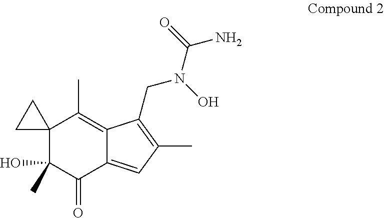 [SMILES: CC1=C(CN(O)C(N)=O)C2=C(C)C3(CC3)[C@@](C)(O)C(=O)C2=C1]

One embodiment includes methods and uses of a compound 1 having Formula I or a pharmaceutically acceptable salt thereof for treating cancer in a subject in need thereof, particularly for treating blood cancers.

In one embodiment, the “subject in need thereof” is a subject having blood cancer. For example, the leukemia is acute myeloid leukemia, acute lymphoblastic leukemia, acute mixed lineage leukemia, myelodysplastic syndrome, a myeloproliferative disorder, or chronic myelogenous leukemia. In certain embodiments the subject is the subject is unable to receive other therapy to treat the cancer due to age or intercurrent illness. In certain embodiments the subject is at least 20 years old, or at least 30 years old, or at least 40 years old, or at least 50 years old, or at least 60 years old, or at least 65 years old, or at least 70 years old or older.

In some embodiments, the subject in need thereof had at least one prior therapy to treat blood cancer such as mantle cell lymphoma (MCL), double-hit lymphoma (DHL), Burkitt's Lymphoma, Anaplastic large cell lymphoma (ALCL), Multiple Myeloma (MM), acute myeloid leukemia (AML), acute lymphoblastic leukemia (ALL), and chronic myeloid leukemia (CML). In some embodiments, the subject has resistant or refractory blood cancers. Resistant or refractory blood cancers are defined as resistant to treatment at the start or acquired resistance during or after the treatment.

In some embodiments, the therapeutically effective amount of hydroxyureamethyl-acylfulvene (e.g.., Compound 1) or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day

The hydroxyureamethyl acylfulvene or its salt may include pharmaceutically acceptable components. The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In one example, hydroxyureamethyl acylfulvene or its salt may be administered either prior to, concomitantly with, or subsequent to the administration of a chemotherapeutic agent or agents.

Contacting a cell with hydroxyureamethyl acylfulvene or Compound 1, or a pharmaceutically acceptable salt, prodrug, metabolite or solvate thereof, can induce or activate cell death selectively in cancer cells. Administering to a subject in need thereof Compound 1, or a pharmaceutically acceptable salt, prodrug, metabolite or solvate thereof, can induce or activate cell death selectively in cancer cells. Contacting a cell with a compound disclosed herein, or a pharmaceutically acceptable salt, prodrug, metabolite, or solvate thereof, can induce cell death selectively in one or more cells affected by a cell proliferative disorder.

In one embodiment, the subject in need thereof can be treated with Compound 1 or a compound disclosed herein as a monotherapy. A “monotherapy” refers to the administration of a single active or therapeutic compound to a subject in need thereof. A monotherapy can involve administration of a therapeutically effective amount of an single active compound.

In another embodiment, any of the above methods of treatment comprises the further step of co-administering to the patient one or more second therapeutic agents. The choice of a combination of agent or second therapeutic agent may be made from any second therapeutic agent known to be useful for co-administration with hydroxyureamethyl acylfulvene or salt. The choice of a second therapeutic agent can dependent upon the particular disease or condition to be treated. Examples of second therapeutic agents that may be employed in the methods of this application are those set forth above for use in combination compositions comprising a compound of this invention and a second therapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as use of other agents and the judgment of the treating physician. For example, guidance for selecting an effective dose can be determined by reference to the prescribing information for hydroxyureamethyl acylfulvene or journal discussion the same.

The term “effective amount” as used herein refers to the amount of an agent needed to alleviate at least one or more symptoms of the disease or disorder, and relates to a sufficient amount of pharmacological composition to provide the desired effect. The term “therapeutically effective amount” therefore refers to an amount of the agent that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Accordingly, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration. Therapeutically effective amounts for a given situation can be determined by routine experimentation that is within the skill and judgment of the clinician. In one aspect, the disease or condition to be treated is CLL. In another aspect, the disease or condition to be treated is AML.

The dosage ranges for the administration of an agent according to the methods described herein depend upon, for example, the form of the agent, its potency, and the extent to which symptoms, markers, or indicators of a condition described herein are desired to be reduced, for example, the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of an agent described herein in, e.g. the treatment of a condition described herein, or to induce a response as described herein (e.g. blood cancer) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size and/or growth rate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. When using an experimental animal model, efficacy of treatment is evidenced when a statistically significant change in a marker is observed, e.g. tumor size and/or growth rate.

The term “treat” is used and includes both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

Treating cancer can result in a reduction in size of a tumor. A reduction in size of a tumor may also be referred to as “tumor regression”. After treatment, tumor size is reduced by 5% or greater relative to its size prior to treatment; or tumor size is reduced by 10% or greater; or reduced by 20% or greater; or reduced by 30% or greater; or reduced by 40% or greater; or reduced by 50% or greater; and or reduced by greater than 75% or greater. Size of a tumor may be measured by any reproducible means of measurement. The size of a tumor may be measured as a diameter of the tumor.

Treating cancer can result in a reduction in tumor volume. After treatment, tumor volume may be reduced by 5% or greater relative to its size prior to treatment; tumor volume may be reduced by 10% or greater; reduced by 20% or greater; reduced by 30% or greater; reduced by 40% or greater; reduced by 50% or greater; or reduced by greater than 75% or greater. Tumor volume may be measured by any reproducible means of measurement.

Treating cancer results in a decrease in number of tumors. After treatment, tumor number may be reduced by 5% or greater relative to number prior to treatment. Further, tumor number may be reduced by 10% or greater; reduced by 20% or greater; reduced by 30% or greater; reduced by 40% or greater; reduced by 50% or greater or reduced by greater than 75%. Number of tumors may be measured by any reproducible means of measurement. The number of tumors may be measured by counting tumors visible to the naked eye or at a specified magnification.

Treating cancer can result in an increase in average survival time of a population of treated subjects in comparison to a population receiving carrier alone. The average survival time can be increased by more than 30 days, by more than 60 days; by more than 90 days; and by more than 120 days. An increase in average survival time of a population may be measured by any reproducible means.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. The average survival time can be increased by more than 30 days; by more than 60 days; by more than 90 days; and by more than 120 days. An increase in average survival time of a population may be measured by any reproducible means. An increase in average survival time of a population may be measured, for example, by calculating for a population the average length of survival following initiation of treatment with an active compound.

Treating cancer can result in a decrease in the mortality rate of a population of treated subjects in comparison to a population receiving carrier alone. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. After treatment, tumor growth rate is reduced by at least 5% relative to number prior to treatment. Further, tumor growth rate can reduced by at least 10%, reduced by at least 20%, reduced by at least 30%, reduced by at least 40%; reduced by at least 50%; reduced by at least 50%; or reduced by at least 75%. Tumor growth rate may be measured by any reproducible means of measurement. Tumor growth rate can be measured according to a change in tumor diameter per unit time.

Treating cancer can result in a decrease in tumor regrowth. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing cancer can result in a decrease in the number or proportion of cells having an abnormal appearance or morphology. An abnormal cellular morphology can be measured by microscopy, e.g., using an inverted tissue culture microscope. An abnormal cellular morphology can take the form of nuclear pleiomorphism.

Treating cancer or a cell proliferative disorder can result in cell death, and cell death results in a decrease of at least 10% in number of cells in a population. Cell death means a decrease of at least 20%; more preferably, a decrease of at least 30%; a decrease of at least 40%; a decrease of at least 50%; or a decrease of at least 75%. Number of cells in a population may be measured by any reproducible means.

The treatment may include the use of one or more biomarkers. The biomarker may be less than, greater than, or equal to the level of those markers in healthy people.

### EXAMPLES

In order that the disclosure disclosed herein may be more efficiently understood, examples are provided below. It should be understood that these examples are for illustrative purposes only and are not to be construed as limiting the disclosure in any manner.

### Example 1

Synthesis of Compound 1 can be found in WO/2020/051222 titled “ILLUDIN ANALOGS, USES THEREOF, AND METHODS FOR SYNTHESIZING THE SAME”.

### Example 2

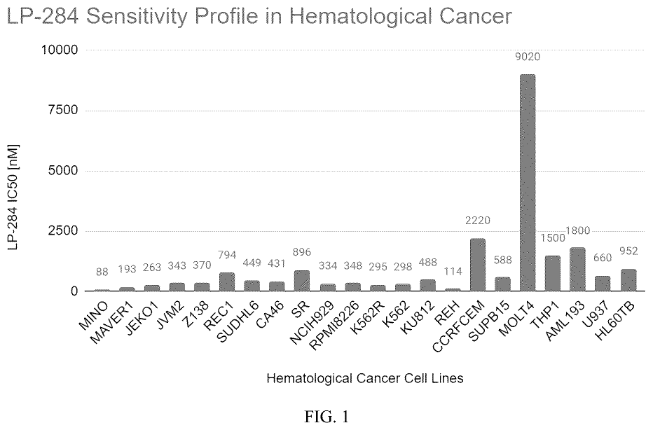

The sensitivity and selectivity of Compound 1 (with positive optical chirality) was assessed using a panel of human blood cell lines. The cell lines used in this Example are shown below and plotted in FIG. 1. The cells were inoculated into 96 well plates at various numbers of cells/well depending on the doubling time of the cell line for 24 hours. Then these cells were treated by Compound 1 at 5˜8 different doses and DMSO vehicle controls. After another 48 or 72 hour incubation, the number viable cells/wells were measured by the sulforhodamine B (SRB) assay (48 h) or the Promega Celltiter Fluor Cell Viability (72 h). IC₅₀ values were then calculated based on normalized cell counts at different Compound 1 doses. Anti-proliferative activity was determined by cell viability measurements. Table 1 shows Compound 1 sensitivity in a variety of Leukemias and Lymphomas. Table 1 shows details of the study.

TABLE 1

| | | LP-284 | Assay |
|---|---|---|---|
| | Type of hematological | IC50 | duration |
| Cell_line | cancer | [nM] | (hours) |
| MINO | Mantle Cell Lymphoma | 88 | 72 |
| MAVER1 | Mantle Cell Lymphoma | 193 | 72 |
| JEKO1 | Mantle Cell Lymphoma | 263 | 72 |
| JVM2 | Mantle Cell Lymphoma | 343 | 72 |
| Z138 | Mantle Cell Lymphoma | 370 | 72 |
| REC1 | Mantle Cell Lymphoma | 794 | 72 |
| SUDHL6 | Double-Hit Lymphoma | 449 | 72 |
| CA46 | Burkitt's Lymphoma | 431 | 72 |
| SR | Anaplastic large cell | 896 | 48 |
| | lymphoma | | |
| NCIH929 | Multiple Myeloma | 334 | 72 |
| RPMI8226 | Multiple Myeloma | 348 | 48 |
| K562R | Chronic Myeloid Leukemia | 295 | 72 |
| K562 | Chronic Myeloid Leukemia | 298 | 72 |
| KU812 | Chronic Myeloid Leukemia | 488 | 72 |
| REH | Acute Lymphoblastic Leukemia | 114 | 72 |
| CCRFCEM | Acute Lymphoblastic Leukemia | 2220 | 48 |
| SUPB15 | Acute Lymphoblastic Leukemia | 588 | 72 |
| MOLT4 | Acute Lymphoblastic Leukemia | 9020 | 48 |
| THP1 | Acute Myeloid Leukemia | 1500 | 72 |
| AML193 | Acute Myeloid Leukemia | 1800 | 72 |
| U937 | Acute Myeloid Leukemia | 660 | 72 |
| HL60TB | Acute Myeloid Leukemia | 952 | 48 |

The foregoing examples and description of the preferred embodiments should be taken as illustrating, rather than as limiting the present invention as defined by the claims.

As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A method for treating a subject diagnosed with blood cancer, the method comprises administering an effective amount of a compound having a structure:

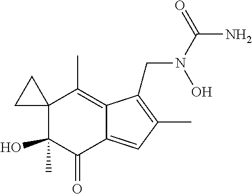 [SMILES: CC1=C(CN(O)C(N)=O)C2=C(C)C3(CC3)[C@](C)(O)C(=O)C2=C1]

2. The method of claim 1, wherein the diagnosed blood cancer is leukemia.

3. The method of claim 1, wherein the diagnosed blood cancer is Mantle Cell Lymphoma.

4. The method of claim 1, wherein the diagnosed blood cancer is Double-Hit Lymphoma.

5. The method of claim 1, wherein the diagnosed blood cancer is an acute leukemia, specifically a relapsed or refractory acute leukemia.

6. The method of claim 1, wherein the diagnosed blood cancer is an acute leukemia, specifically either relapsed or refractory acute lymphoblastic leukemia (ALL) or relapsed or refractory acute myelogenous leukemia (AML).

7. The method of claim 1, wherein the diagnosed blood cancer is lymphoma or myeloma.

8. The method of claim 1, wherein the diagnosed blood cancer is one of the following: mantle cell lymphoma (MCL), double-hit lymphoma (DHL), Burkitt's Lymphoma, Anaplastic large cell lymphoma (ALCL), or Multiple Myeloma (MM).

9. The method of claim 1, which also includes administering a second anti-cancer agent to the subject.

10. The method of claim 9, wherein the second anti-cancer agent is chosen from DNA damage agents, glucocorticoids, immunomodulatory drugs (IMiDs), BCL2 inhibitors, Bruton's tyrosine kinase inhibitors, spironolactone, PARP inhibitors, and proteasome inhibitors.

11. The method of claim 1, wherein the subject is concurrently undergoing another therapy for conditions such as acute myeloid leukemia, acute lymphoblastic leukemia, myelodysplastic syndrome, a myeloproliferative disorder, or chronic myelogenous leukemia.

12. The method of claim 1, wherein the subject is a human.

13. The method of claim 1, detailing the administration method, which can be oral, topical, intranasal, systemic, intravenous, subcutaneous, intraperitoneal, intradermal, intraocular, iontophoretic, transmucosal, or intramuscular.

14. The method of claim 1, specifying that the compound is introduced either intravenously or intraperitoneally.

15. The method of claim 1, wherein the diagnosed blood cancer is from the group including: mantle cell lymphoma (MCL), double-hit lymphoma (DHL), Burkitt's Lymphoma, Anaplastic large cell lymphoma (ALCL), Multiple Myeloma (MM), acute myeloid leukemia (AML), acute lymphoblastic leukemia (ALL), and chronic myeloid leukemia (CML).

16. A method for treating a subject with blood cancer using an effective amount of hydroxyureamethyl acylfulvene.

17. The method of claim 16, the hydroxyureamethyl acylfulvene has the following structure:

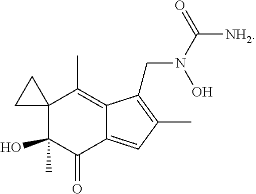 [SMILES: CC1=C(CN(O)C(N)=O)C2=C(C)C3(CC3)[C@](C)(O)C(=O)C2=C1]

18. The method of claim 16, wherein the subject is concurrently receiving therapy for one of the following conditions: mantle cell lymphoma (MCL), double-hit lymphoma (DHL), Burkitt's Lymphoma, Anaplastic large cell lymphoma (ALCL), Multiple Myeloma (MM), acute myeloid leukemia (AML), acute lymphoblastic leukemia (ALL), or chronic myeloid leukemia (CML).

19. The method of claim 16, wherein the diagnosed blood cancer is Mantle Cell Lymphoma.

20. The method of claim 16, wherein the diagnosed blood cancer is Double-Hit Lymphoma.
